(19) United States
(12) Patent Application Publication
(10) Pub. No.: US 2022/0162164 A1
(43) Pub. Date: May 26, 2022

(54) PROCESSES FOR PREPARING ALPHA-CARBOXAMIDE PYRROLIDINE DERIVATIVES

(71) Applicant: Biongen Inc., Cambridge, MA (US)

(72) Inventors: Masafumi Nakanishi, Kanagawa (JP); Yoshihiko Usui, Kanagawa (JP); Takuya Kawai, Kanagawa (JP); Tomohiro Ishizaka, Kanagawa (JP)

(21) Appl. No.: 17/602,651

(22) PCT Filed: Apr. 9, 2020
(86) PCT No.: PCT/US2020/027460

§ 371 Date: Oct. 8, 2021

Related U.S. Application Data
(60) Provisional application No. 62/831,980, filed on Apr. 10, 2019.

(51) Int. Cl. C07D 207/16

(52) U.S. Cl. CPC C07D 207/16

(57) ABSTRACT

The disclosure provides processes for preparing α-carboxamide pyrrolidine derivatives, in particular (2S,5R)-5-(4-((2-fluorobenzyl)oxy)phenyl)pyrrolidine-2-carboxamide, as well as intermediates for use in said processes.

### RELATED APPLICATIONS

This application claims the benefit of U.S. Provisional Application No. 62/831,980, filed Apr. 10, 2019, the contents of which are incorporated herein by reference in their entirety.

### BACKGROUND OF THE INVENTION

(2S,5R)-5-(4-((2-fluorobenzyl)oxy)phenyl)pyrrolidine-2-carboxamide:

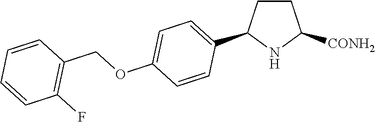 [SMILES: NC(=O)[C@@H]1CC[C@H](c2ccc(OCc3ccccc3F)cc2)N1]

is described in U.S. Pat. No. 7,655,693 as having utility in the treatment of diseases and conditions mediated by modulation of use-dependent voltage-gated sodium channels. Certain synthetic methods to prepare (2S,5R)-5-(4-((2-fluorobenzyl)oxy)phenyl)pyrrolidine-2-carboxamide are described in U.S. Pat. Nos. 7,655,693 and 8,759,542. The contents of each of these patents are incorporated by reference in their entirety.

However, there is a need for the development of alternative processes for the preparation of such α-carboxamide pyrrolidine derivatives, which are capable of practical application to large scale manufacture.

### SUMMARY OF THE INVENTION

The present invention provides processes for preparing a compound of formula (I)

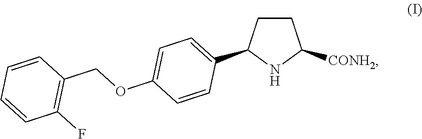 [SMILES: NC(=O)[C@@H]1CC[C@H](c2ccc(OCc3ccccc3F)cc2)N1]

or a pharmaceutically acceptable salt thereof, comprising reacting a compound of formula (III) with a compound of formula (4), thereby producing a compound of formula (IV):

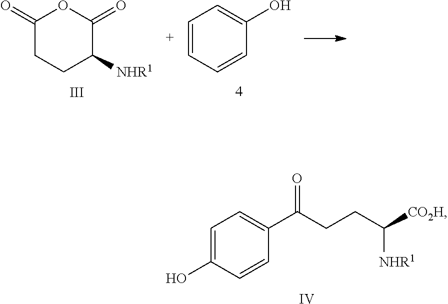

wherein R¹ is a resonance-accepting nitrogen-protecting group.

### DETAILED DESCRIPTION

In certain aspects, the present disclosure provides processes for preparing a compound of formula (I)

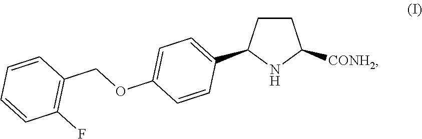 [SMILES: NC(=O)[C@@H]1CC[C@H](c2ccc(OCc3ccccc3F)cc2)N1]

or a pharmaceutically acceptable salt thereof, comprising reacting a compound of formula (III) with a compound of formula (4), thereby producing a compound of formula (IV):

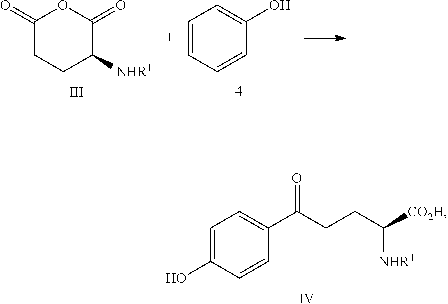

wherein R¹ is a resonance-accepting nitrogen-protecting group, e.g., a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps, and trifluoroacetyl. In certain preferred embodiments, R¹ is trifluoroacetyl. In certain embodiments, the process is for producing a compound of formula (IV).

A “resonance-accepting nitrogen-protecting group”, as the term is used herein, refers to a protecting group that has a x orbital (e.g., an orbital participating in a double or triple bond) capable of accepting electron density from the lone pair of the nitrogen atom to which it is attached, e.g., via a resonance form or tautomer. Carbonyl moieties (e.g., as present in amide, urea, and carbamate functional groups) and sulfonyl moieties (e.g., as present in sulfonamide functional groups) are representative groups capable of accepting electron density from the nitrogen atom in those functional groups.

In certain embodiments, reacting the compound of formula (III) with the compound of formula (4) comprises reacting the compound of formula (III) with the compound of formula (4) in the presence of a metal salt (such as aluminum salt, e.g., aluminum trichloride) and a solvent (such as nitrobenzene).

In certain embodiments, the processes described herein comprise reacting compound (1) with a compound that provides a nitrogen-protecting group, thereby producing a compound of formula (II):

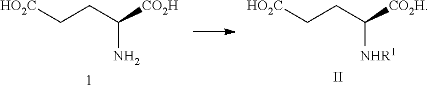

Any suitable reaction to provide a nitrogen-protecting group may be used. In certain embodiments, reacting compound (1) with a compound that provides a nitrogen-protecting group comprises reacting compound (1) with a compound that provides a nitrogen-protecting group (e.g., ethyl trifluoroacetate) in the presence of a solvent, such as methanol. In certain embodiments, the reaction is performed in the presence of an amine base, such as triethylamine.

In certain embodiments, the processes described herein comprise dehydrating the compound of formula (II), thereby producing the compound of formula (III):

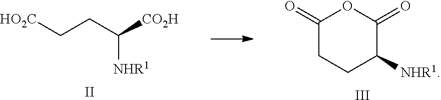

In certain embodiments, dehydrating the compound of formula (II) comprises reacting the compound of formula (II) with acetic anhydride.

In certain embodiments, the processes described herein comprise deprotecting the compound of formula (IV), thereby producing a compound of formula (V):

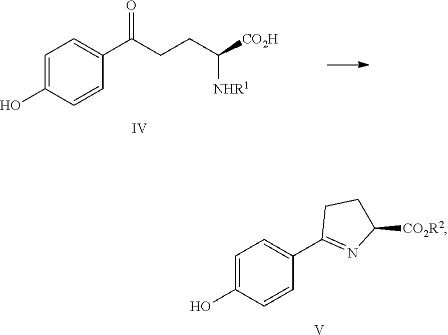

wherein R² is C₁₋₆ alkyl, C₂₋₆ alkenyl, or C₂₋₆ alkynyl.

In certain aspects, the present disclosure provides processes for preparing a compound of formula (I)

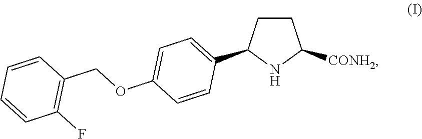 [SMILES: NC(=O)[C@@H]1CC[C@H](c2ccc(OCc3ccccc3F)cc2)N1]

or a pharmaceutically acceptable salt thereof, comprising deprotecting the compound of formula (IV), thereby producing a compound of formula (V).

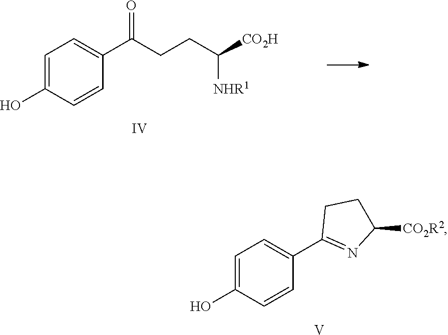

wherein R² is C₁₋₆alkyl, C₂₋₆ alkenyl, or C₂₋₆ alkynyl. In certain embodiments, the process is for producing a compound of formula (V).

In certain embodiments, deprotecting the compound of formula (V) comprises reacting the compound of formula (IV) with an acid (such as sulfuric acid), e.g., in the presence of a solvent, such as methanol.

In certain embodiments, the processes described herein comprise reacting the compound of formula (V) with hydrogen gas and a compound that provides a nitrogen-protecting group in the presence of a catalyst, thereby producing a compound of formula (VI):

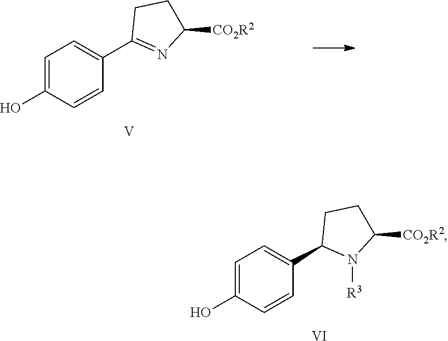

wherein R³ is a resonance-accepting nitrogen-protecting group, e.g., a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps; and trifluoroacetyl. In certain preferred embodiments, R³ is tert-butyloxycarbonyl (Boc).

Any suitable reaction to provide a nitrogen-protecting group may be used. In certain embodiments, reacting the compound of formula (V) comprises reacting the compound of formula (V) with hydrogen gas and the compound that provides a nitrogen-protecting group (such as di-tert-butyldicarbonate) in the presence of the catalyst, and e.g., a solvent, such as methanol. In certain embodiments, the catalyst is Pd/C, such as 5% Pd/C.

In certain embodiments, R² is C₁₋₆alkyl, preferably methyl.

In certain embodiments, the processes comprise:

reacting a compound (1) with the compound that provides a nitrogen-protecting group, thereby producing a compound of formula (II):

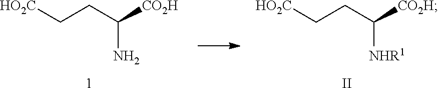

dehydrating the compound of formula (II), thereby producing a compound of formula (III):

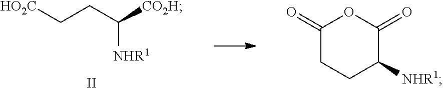

reacting the compound of formula (III) with a compound of formula (4), thereby producing a compound of formula (IV):

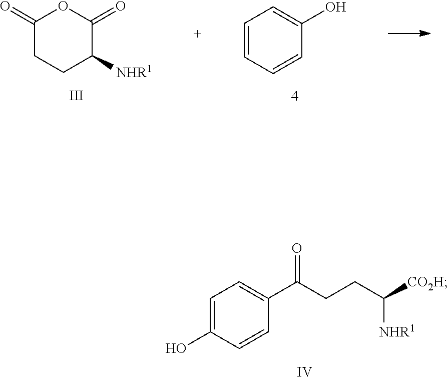

deprotecting the compound of formula (IV), thereby producing a compound of formula (V):

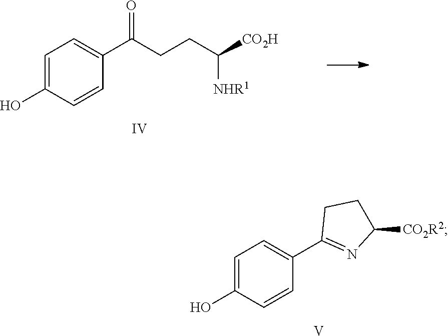

reacting the compound of formula (V) with hydrogen gas and a compound that provides a nitrogen-protecting group in the presence of the catalyst, thereby producing a compound of formula (VI):

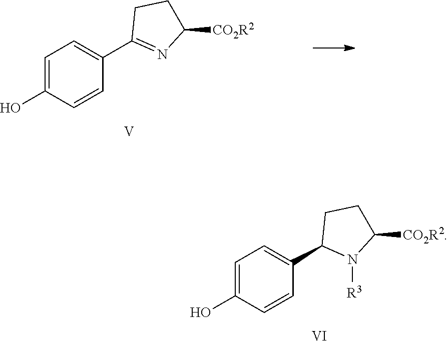

In certain aspects, the present disclosure provides compounds of formula (IV)

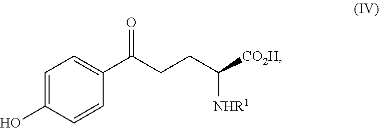

or a salt thereof, wherein R¹ is a resonance-accepting nitrogen-protecting group, e.g., a nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps, and trifluoroacetyl.

In certain embodiments, the compounds of formula (IV) is of the structure

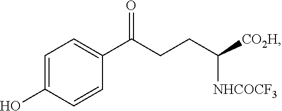

or a salt thereof.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)z. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

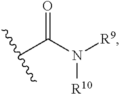

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

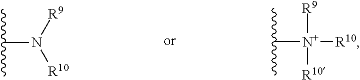

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

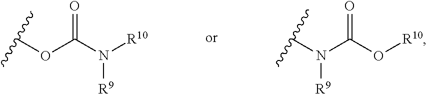

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

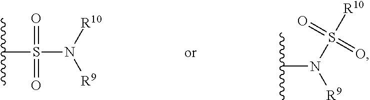

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

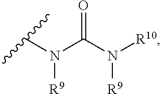

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Salt” is used herein to refer to an acid addition salt or a basic addition salt.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

The term “steroid” as used herein refers to naturally occurring and synthetic compounds, based on the cyclopenta[a]phenanthrene carbon skeleton, that may be partially or completely saturated. It will be understood by those skilled in the art that the carbon skeleton can be substituted, if appropriate. Examples of steroids include, but are not limited to, alclometasone, prednisone, dexamethasone, triamcinolone, cortisone, fludrocortisone, dihydrotachysterol, oxandrolone, oxabolone, testosterone, nandrolone, diethylstilbestrol, estradiol, norethisterone, medroxyprogesterone acetate, hydroxyprogesterone caproate.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Exemplary Materials and Methods

Unless otherwise stated, reactions were performed in jacketed glass-lined reactors under a nitrogen atmosphere. All solvents and reagents were used as received from commercial sources, unless otherwise noted. Reaction temperatures above 23° C. refer to jacket temperatures. ¹H and ¹³C NMR spectra were recorded using Bruker AV-500, DRX-500, and AV-400 MHz spectrometers, with ¹³C NMR spectroscopic operating frequencies of 125, 125, and 100 MHz, respectively. Chemical shifts (5) are reported in parts per million (ppm) relative to the residual protonated solvent: CDCl₃ signal (δ=7.26 for ¹H NMR; δ=77.2 for ¹³C NMR), C₆D₆ signal (δ =7.16 for ¹H NMR; δ=128.1 for ¹³C NMR), DMSO-d₆ (δ=2.50 for ¹H NMR; δ=39.5 for ¹³C NMR). Data for ¹H NMR spectra are reported as follows: chemical shift, multiplicity, coupling constants (Hz), and number of hydrogen atoms. Data for ¹³C NMR spectra are reported in terms of chemical shift. The following abbreviations are used to describe the multiplicities: s=singlet; d=doublet; t=triplet; q=quartet; quint=quintet; m=multiplet; br=broad. Melting points (MP) are uncorrected and were recorded using an Electrothermal® capillary melting point apparatus. IR spectra were recorded on a Jasco FITR-4100 spectrometer with an ATR attachment, the selected signals are reported in cm-1. HRMS (DART) was performed using a Thermo Fisher Scientific Exactive Plus spectrometer equipped with an IonSense ID-CUBE DART source. X-ray crystallographic data were collected using a Bruker SMART CCD-based diffractometer equipped with a low-temperature apparatus operated at 100 K.

### Example 1: Preparation of (2,2,2-Trifluoroacetyl)-L-Glutamic Acid

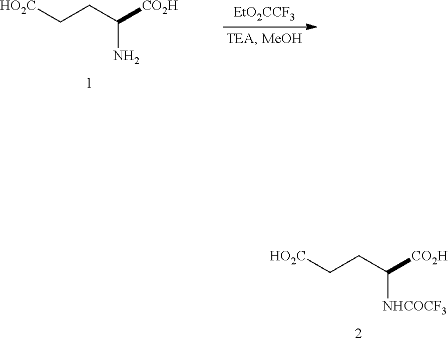

Procedure A

A reactor was charged with L-glutamic acid (86 kg), MeOH (5V) and triethylamine (2 eq). Ethyl trifluoroacetate (1.3 eq) was added with good agitation while maintaining a reaction temperature of 15-30° C. The contents of the reactor were maintained at 20-30° C.; the progress of the reaction was monitored for completion (HPLC). The reaction was concentrated in vacuo to low volume, water (5V) was charged and the contents of the reactor were cooled. Concentrated aqueous HCl solution (136 wt %) was added with good agitation while maintaining a reaction temperature of 5-15° C. EtOAc (8.8V) was charged, the mixture was agitated for about 15 min and the layers were separated. The aqueous layer was extracted with EtOAc (4.4V). The combined organic layers were washed with water (3.5V), dried over anhydrous Na₂SO₄ (58 wt %) and filtered The spent filter cake was washed with EtOAc (0.6V) and the combined filtrate and wash was concentrated in vacuo to low volume. Petroleum ether (6.2V) was charged to the resulting residue and the mixture was cooled to −10 to −20° C. The solids were isolated by filtration, washed with petroleum ether (1.4V) and dried in vacuo at 35-42° C. to constant weight to give 129.99 kg (91%) of the title compound.

A portion of the lot (9.95 kg) was agitated for about 2 h in water (5V), the mixture was filtered and the filter cake was washed with water (3V); 6.6 kg (66% recovery) of the title compound was obtained. The wet solid was dissolved in EtOAc (13.6V), washed twice with water (7.6V) and the organic layer was concentrated in vacuo to low volume. Petroleum ether (8.4V) was charged to the resulting residue and the resulting mixture was cooled to −10 to −20° C. The solids were isolated by filtration and dried in vacuo at 35-42° C. to constant weight to give 5.77 kg (58% recovery) of the title compound.

Procedure B

A reactor was charged with L-glutamic acid (14 kg), MeOH (5V) and triethylamine (2 eq). Ethyl trifluoroacetate (1.3 eq) was added with good agitation while maintaining a reaction temperature of 15-30° C. The contents of the reactor were maintained at 20-30° C.; the progress of the reaction was monitored for completion (HPLC). The reaction was concentrated in vacuo to low volume, water (5V) was charged and the contents of the reactor were cooled. Concentrated aqueous HCl solution (222 wt %) was added with good agitation while maintaining a reaction temperature of 5-15° C. and the mixture was aged for about 2 h. Solids were isolated by filtration, and the filter cake was washed with water (5.7V) and dried in vacuo at 35-42° C. to constant weight to give 18.90 kg (82%) of the title compound.

The solids were dissolved in EtOAc (10.3V), washed twice with water (2.6V) and the organic layer was dried over anhydrous Na₂SO₄ (159 wt %) and filtered. The spent filter cake was washed with EtOAc (1.2V) and the combined filtrate and wash was concentrated in vacuo to low volume. Petroleum ether (5.7V) was charged to the resulting residue and the mixture was cooled to −10 to −20° C. The solids were isolated by filtration, washed with petroleum ether (2.5V) and dried in vacuo at 35-42° C. to constant weight to give 15.4 kg (81% recovery) of the title compound.

The solids were dissolved in EtOAc (5.8V), washed twice with water (3.2V) and the organic layer was concentrated in vacuo to low volume. Petroleum ether (6.1V) was charged to the resulting residue and the mixture was cooled to −10 to −20° C. The solids were isolated by filtration, washed with petroleum ether (2.5V) and dried in vacuo at 35-42° C. to constant weight to give 13.0 kg (84% recovery) of the title compound.

### Example 2: Preparation of (S)—N-(2,6-dioxotetrahydro-2H-pyran-3-yl)-2,2,2-trifluoroacetamide

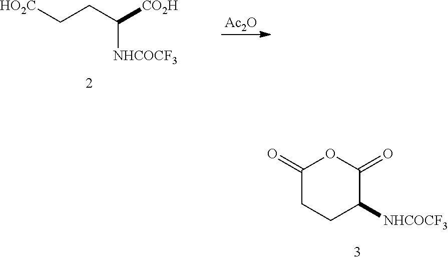

A reactor was charged with (2,2,2-trifluoroacetyl)-L-glutamic acid (18.8 kg) and acetic anhydride (5.1 kg). The contents of the reactor were heated to 70-80° C.; the progress of the reaction was monitored for completion (benzylamine derivatization; HPLC). The contents of the reactor were cooled to 30-40° C. and concentrated in vacuo at <40° C. to low volume. To the resulting residue was charged MTBE (2.7V), the solution was cooled to induce crystallization, and the contents of the reactor were further cooled to −10 to −20° C. and aged for about 2 h. The solids were isolated by filtration and washed with MTBE (1.8V) to give 2.9 kg (17%) of the title compound.

The combined filtrate and wash were concentrated in vacuo to low volume, and the resulting residue was twice reconcentrated from toluene (0.92V) in vacuo at <40° C. To the resulting residue was charged MTBE (1.6V), the solution was cooled to induce crystallization, and the contents of the reactor were further cooled to −10 to −20° C. and aged for about 2 h. The solids were isolated by filtration and washed with MTBE (1.4V) to give 4.1 kg (24%) of the title compound.

The solids from both runs were combined and dried in vacuo to constant weight to give 6.34 kg (36%) of the title compound.

### Example 3: Preparation of (S)-5-(4-hydroxyphenyl)-5-oxo-2-(2,2,2-trifluoroacetamido)pentanoic Acid

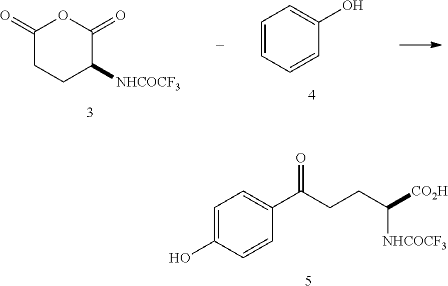

A reactor was charged with AlCl₃ (2.5 eq) and nitrobenzene (4V). (S)—N-(2,6-dioxotetrahydro-2H-pyran-3-yl)-2,2,2-trifluoroacetamide (6.34 kg) was charged while maintaining a reaction temperature of 25-35° C. After aging for about 1 h, a solution of phenol (1.5 eq) in nitrobenzene (IV) was added while maintaining a reaction temperature of 25-35° C. Following the addition, the contents of the reactor were aged for about 2 h, then heated to and maintained at 75-80° C.; the progress of the reaction was monitored for completion (HPLC). The contents of the reactor were cooled to 25-35° C. and quenched into pre-cooled water (0-10° C.; 10V). Concentrated aqueous HCl (100 wt %) and EtOAc (7V) were charged with good agitation, and the layers were split. The organic layer was washed with brine (11.8 wt), dried over anhydrous Na₂SO₄ (3 wt) and filtered. The spent filter cake was washed with EtOAc (2.2V) and the combined filtrate and wash were concentrated in vacuo to low volume. The mixture was cooled to 15-20° C., aged for about 1 h and toluene (5V) was charged. The mixture was aged for about 2 h and solids were collected by filtration and washed with toluene (2V). The solids were agitated for about 2 h in toluene (7.3V) at 15-20° C., filtered, washed with toluene (4.9V) and dried in vacuo at 40-45° C. to constant weight to give 5.3 kg (59%) of the title compound.

### Example 4: Preparation of Methyl (S)-5-(4-hydroxyphenyl)-3,4-dihydro-2H-pyrrole-2-carboxylate

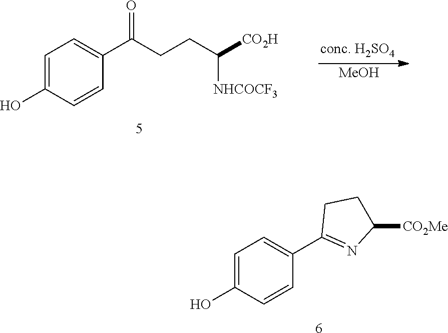

A reactor was charged with (S)-5-(4-hydroxyphenyl)-5-oxo-2-(2,2,2-trifluoroacetamido)pentanoic acid (5.3 kg), activated carbon (22.6 wt %) and MeOH (7.6V). The mixture was agitated at ambient temperature for about 1 h, filtered and the filter cake was washed with MeOH (2.5V). To the combined filtrate and wash was charged concentrated H₂SO₄ (98%; 9.4 eq) and the solution was heated to and maintained at 65-75° C.; the progress of the reaction was monitored for completion (HPLC). The contents of the reactor were cooled to 25-35° C. and were concentrated in vacuo to low volume. To the resulting residue was charged a solution prepared from NaHCO₃(5 wt) and water (51.7V) while maintaining a reaction temperature of 10-20° C. The contents of the reactor were aged at 20-30° C. for about 2 h. The solids were filtered, washed with water (3.8V) and dried in vacuo at 35-45° C. to constant weight to give 1.1 kg (30%) of the title compound.

A reactor was charged with methyl (S)-5-(4-hydroxyphenyl)-3,4-dihydro-2H-pyrrole-2-carboxylate (1.07 kg) and MTBE (5V). The mixture was heated to and maintained at about 35° C. for about 2 h, cooled to about 2° C. and filtered. The wet solids were dried in vacuo at 30° C. to constant weight to give 0.98 kg (92% recovery) of the title compound.

### Example 5: Preparation of 1-(tert-butyl) 2-methyl (2S,5R)-5-(4-hydroxyphenyl)pyrrolidine-1,2-dicarboxylate

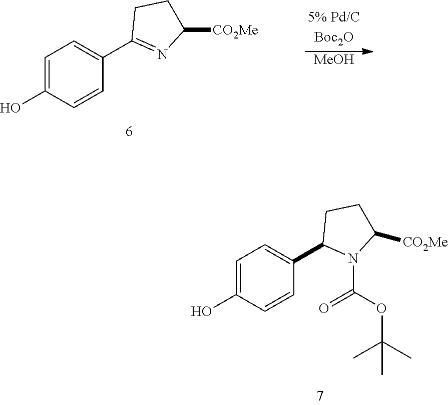

A 5 L hydrogenation autoclave pre-cooled to 5° C. was charged with a solution of di-tert-butyldicarbonate (0.98 eq) in MeOH (2V), a suspension of activated carbon (10 wt %) in MeOH (1V), a suspension of 5% Pd/C (6 wt %) in MeOH (1V) and a suspension of methyl (S)-5-(4-hydroxyphenyl)-3,4-dihydro-2H-pyrrole-2-carboxylate (350 g) in MeOH (5V). With good agitation, multiple vacuum/nitrogen pressurization cycles and multiple vacuum/hydrogen pressurization cycles were performed. The contents of the autoclave were placed under hydrogen pressure (10 bar); the reaction was performed at 22-27° C. The progress of the reaction was monitored for completion (HPLC). MeOH (4V) was charged, the reaction mixture was passed through a bed of filter aid and the filtrate was concentrated in vacuo to a net weight of about 510 wt % (about 9V of distillate collected). The resulting mixture was cooled to 25° C., water (2.8V) was added over about 30 min and the mixture was cooled to 0-5° C. The solids were filtered, washed twice with 20% MeOH in water (3V), once with MTBE (2V) and dried in vacuo at about 55° C. to constant weight to give 421 g (82%) of the title compound.

### Example 6: Preparation of (2,2,2-trifluoroacetyl)-L-glutamic Acid

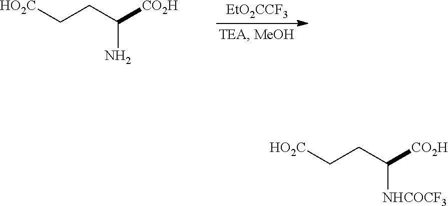

A reactor was charged with L-glutamic acid (174.2 kg) and MeOH (4.1V). Triethylamine (2.0 eq) was added while maintaining a temperature of 20-30° C. Ethyl trifluoroacetate (1.3 eq) was added while maintaining a reaction temperature of 20-30° C. The contents of the reactor were maintained at 20-30° C. with good agitation; the progress of the reaction was monitored for completion (HPLC). Water (6V) was charged while maintaining a reaction temperature of <42° C. The contents of the reactor were concentrated in vacuo to about 5.1× the input L-glutamic acid weight. To the resulting residue was charged water (7V) and concentrated aqueous HCl solution (3.2 eq) with good agitation while maintaining a reaction temperature of 20-25° C. The mixture was stirred for 2 h; solids were collected by filtration. The filter cake was slurried in water (2.5V) at 20-25° C. Solids were collected by filtration, washed with water (1.5V) and dried in vacuo (<10 mmHg) at 30-50° C. to constant weight (KF≤1.0%) to give 224.3 kg (78%) of the title compound.

### Example 7: Preparation of (S)—N-(2,6-dioxotetrahydro-2H-pyran-3-yl)-2,2,2-trifluoroacetamide

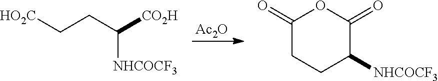

A reactor was charged with acetic anhydride (5.0 eq) and (2,2,2-trifluoroacetyl)-L-glutamic acid (224.0 kg). The contents of the reactor were heated to 65-70° C.; the progress of the reaction was monitored for completion (HPLC). The contents of the reactor were cooled to 30-40° C. and concentrated in vacuo (<10 mmHg) at ≤50° C. until the rate of distillation slowed significantly. To the resulting residue was charged MTBE (2.0V) and the solution was cooled to 8-12° C. to induce crystallization. Toluene (8.0V) was charged and the contents of the reactor were aged for about 1 h at 8-12° C. The slurry was cooled to −10 to −15° C. and aged for about 2 h. The solids were isolated by filtration, washed with cold toluene (4.0V; −10 to −15° C.) and dried to constant weight in vacuo (<12 mmHg) at 35-40° C. to give 170 kg (82%) of the title compound.

### Example 8: Preparation of (S)-5-(4-hydroxyphenyl)-5-oxo-2-(2,2,2-trifluoroacetamido)pentanoic Acid

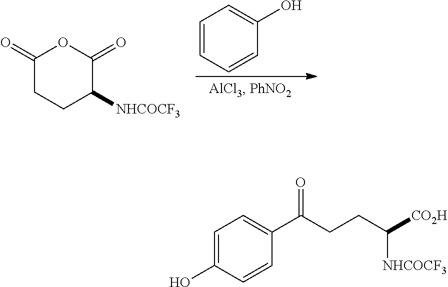

A reactor was charged with nitrobenzene (4.0V) and AlCl₃ (2.0 eq) and the contents of the reactor were stirred at 20-30° C. to give a solution. (S)—N-(2,6-dioxotetrahydro-2H-pyran-3-yl)-2,2,2-trifluoroacetamide (170.5 kg) was charged while maintaining a reaction temperature of 15-25° C. A solution of phenol (1.5 eq) in nitrobenzene (1V) was added while maintaining a reaction temperature of 15-25° C. Following the addition, the contents of the reactor were aged for about 2 h, then heated to and maintained at 75-80° C.; the progress of the reaction was monitored for completion (HPLC). The contents of the reactor were cooled to 30-40° C. and quenched into a mixture of water (10.0V), concentrated aqueous HCl (2.2 eq) and MTBE (7.1V) with good agitation while maintaining a temperature of 20-45° C., and the mixture was stirred for about 30 min at 40-45° C. The layers were split and the organic layer was stirred with 20% brine solution (10.0 wt) for about 30 min at 40-45° C. The layers were split, and the organic layer was stirred with activated carbon (10 wt %) for about 2 h, filtered and the spent filter cake was washed with MTBE (2.0V). The combined filtrate and wash were concentrated in vacuo (<35 mmHg) at ≤40° C. until the rate of distillation slowed significantly. The resulting residue was warmed to 35-45° C., seeded (0.1 wt %) and the contents of the reactor were agitated for about 1 h. Toluene (5.0V′) was charged and agitation was continued at 35-45° C. for about 2 h. The contents of the reactor were cooled to 20-30° C. and aged for about 5 h. Solids were collected by filtration, washed with toluene (4.0V) and dried in vacuo (<10 mmHg) at 45-50° C. to constant weight to give 122 kg (51%) of the title compound.

### Example 9: Preparation of Methyl (S)-5-(4-hydroxyphenyl)-3,4-dihydro-2H-pyrrole-2-carboxylate

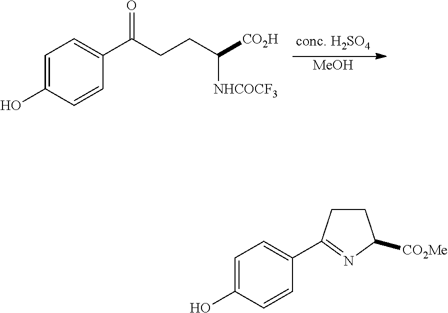

A reactor was charged with MeOH (10.0V) and (S)-5-(4-hydroxyphenyl)-5-oxo-2-(2,2,2-trifluoroacetamido)pentanoic acid (122.8 kg). The mixture was agitated and cooled to 0-10° C. and concentrated H₂SO₄ (9.4 eq) was added while maintaining a reaction temperature of 0-10° C. The contents of the reactor was heated to and maintained at 65-70° C.; the progress of the reaction was monitored for completion (HPLC). The contents of the reactor were cooled to 25-35° C. and were concentrated in vacuo (≤15 mmHg) at <45° C. until the rate of distillation slowed significantly. The resulting residue was charged into a solution prepared from NaHCO₃ (13 eq) and water (24.9V). The contents of the reactor were seeded (0.1 wt %) and agitation was continued at 20-25° C. for about 2 h. Solids were filtered, washed with water (4.1V) and dried in vacuo (≤10 mmHg) at 45-50° C. to constant weight to give 56.92 kg (68%) of the title compound.

A reactor was charged with MeOH (3.0V) and concentrated H₂SO₄ (2.0 eq) while maintaining a temperature of <40° C. Methyl (S)-5-(4-hydroxyphenyl)-3,4-dihydro-2H-pyrrole-2-carboxylate (55.40 kg) was charged and the contents of the reactor were aged at ≤15° C. for about 1 h. The contents of the reactor were charged into a solution prepared from NaHCO₃ (4.0 eq) and water (25V) while maintaining a reaction temperature of 15-25° C. The mixture was aged for about 1 h. Solids were collected by filtration, washed with water (4.0V) and dried in vacuo (≤20 mmHg) at 45-55° C. to constant weight to give 49.35 kg (89% recovery) of the title compound.

### Example 10: Preparation of 1-(tert-butyl) 2-methyl (2S,5R)-5-(4-hydroxyphenyl)pyrrolidine-1,2-dicarboxylate

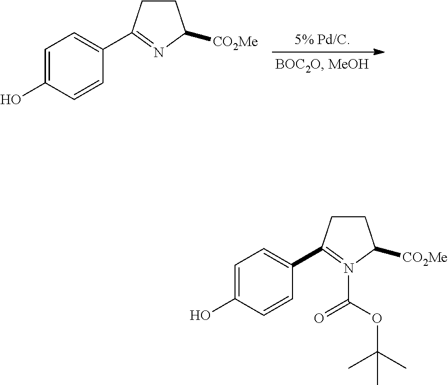

A pre-cooled hydrogenation autoclave was charged with MeOH (5.0V) and methyl (S)-5-(4-hydroxyphenyl)-3,4-dihydro-2H-pyrrole-2-carboxylate (49.28 kg) while maintaining a temperature of <10° C. The autoclave was charged with a suspension composed of water wet 5% Pd/C (4 wt %) and MeOH (2.0V) followed by a solution of di-tert-butyldicarbonate (1.00 eq) in MeOH (2V). With good agitation, multiple vacuum/nitrogen pressurization cycles and multiple vacuum/hydrogen pressurization cycles were performed. The contents of the autoclave were placed under hydrogen pressure (9 bar) and the contents of the autoclave were warmed to 22° C. The progress of the reaction, maintained at <40° C. and 9-11 bar, was monitored for completion (HPLC). MeOH (8V) was charged and the mixture was aged at about 45° C. for about 30 min. The reaction mixture was filtered and Radiolite (5.0 kg) was charged to the filtrate. The mixture was stirred for about 15 min, filtered and the filtrate was concentrated in vacuo to a net weight of about 505 wt %. The resulting mixture was cooled to 30° C., water (2.8V) was added over about 1 h and the mixture was aged at 30° C. for about 30 min. The contents of the reactor were cooled to −5-5° C. and were aged for about 80 min. The solids were filtered, washed with 20% MeOH in water (2V) and dried in vacuo (<20 mmHg) at about 55° C. to constant weight to give 64.32 kg (89%) of the title compound.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A process for preparing a compound of formula (I) 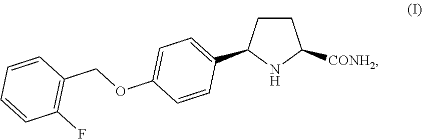 [SMILES: NC(=O)[C@@H]1CC[C@H](c2ccc(OCc3ccccc3F)cc2)N1]
or a pharmaceutically acceptable salt thereof, comprising reacting a compound of formula (III) with a compound of formula (4), thereby producing a compound of formula 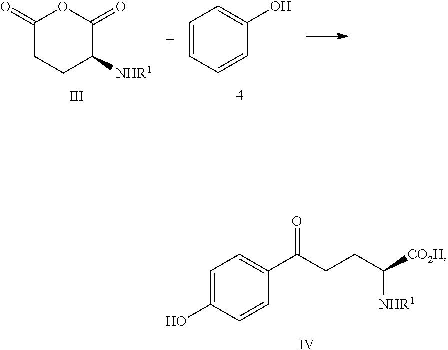
wherein R¹ is a resonance-accepting nitrogen-protecting group.

2. The process of claim 1, comprising reacting the compound of formula (III) and the compound of formula (4) with a metal salt and a solvent.

3. The process of claim 2, wherein the metal salt is an aluminum salt.

4. The process of claim 3, wherein the aluminum salt is aluminum trichloride.

5. The process of any one of claims 2-4, wherein the solvent is nitrobenzene.

6. The process of any one of the preceding claims, further comprising reacting compound (I) with a compound that provides a nitrogen-protecting group, thereby producing a compound of formula (II):

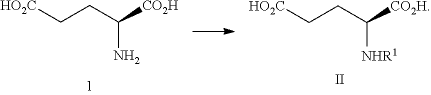

7. The process of claim 6, comprising reacting the compound (1) with the compound that provides a nitrogen-protecting group in the presence of a solvent, and optionally in the presence of an amine base.

8. The process of claim 6 or 7, wherein the compound that provides a nitrogen-protecting group is ethyl trifluoroacetate.

9. The process of any one of claims 6-8, wherein the solvent is methanol, and further wherein the amine base, when present, is triethylamine.

10. The process of any one of claims 6-9, further comprising dehydrating the compound of formula (II), thereby producing the compound of formula (III):

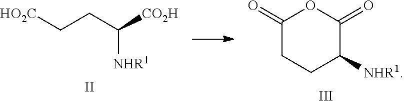

11. The process of claim 10, wherein dehydrating the compound of formula (II) comprises reacting the compound of formula (II) with acetic anhydride.

12. The process of any one of the preceding claims, further comprising deprotecting the compound of formula (IV), thereby producing a compound of formula (V): 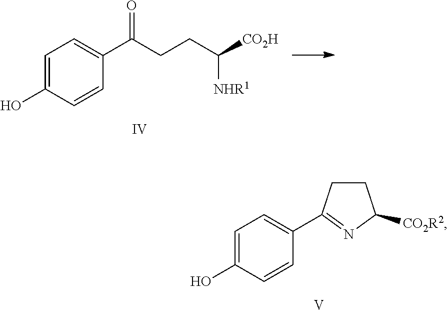
wherein R² is C₁₋₆ alkyl, C₂₋₆ alkenyl, or C₂₋₆ alkynyl.

13. A process for preparing a compound of formula (I) 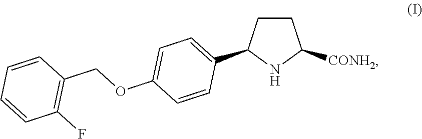 [SMILES: NC(=O)[C@@H]1CC[C@H](c2ccc(OCc3ccccc3F)cc2)N1]
or a pharmaceutically acceptable salt thereof, comprising deprotecting a compound of formula (IV), thereby producing a compound of formula (V) 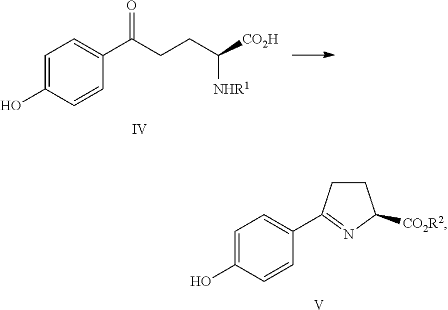
wherein:
R¹ is a resonance-accepting nitrogen-protecting group; and
R² is C₁₋₆ alkyl, C₂₋₆ alkenyl, or C₂₋₆ alkynyl.

14. The process of claim 12 or 13, wherein deprotecting the compound of formula (IV) comprises reacting the compound of formula (IV) with an acid.

15. The process of any one of claims 12-14, wherein deprotecting the compound of formula (IV) comprises reacting the compound of formula (IV) with an acid in the presence of a solvent.

16. The process of claim 14 or 15, wherein the acid is sulfuric acid.

17. The process of claim 15 or 16, wherein the solvent is methanol.

18. The process of any one of the preceding claims, wherein R¹ is a resonance-accepting nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps; and trifluoroacetyl.

19. The process of any one of the preceding claims, wherein R¹ is trifluoroacetyl.

20. The process of any one of claims 12-19, further comprising reacting the compound of formula (V) with hydrogen gas and a compound that provides a nitrogen-protecting group in the presence of a catalyst, thereby producing a compound of formula (VI): 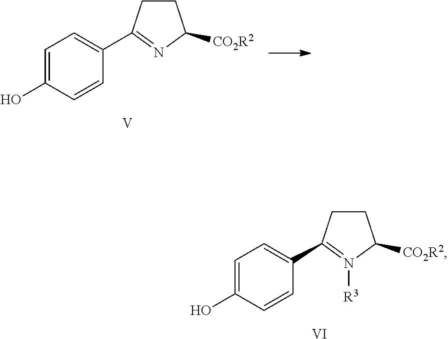
wherein R³ is a resonance-accepting nitrogen-protecting group.

21. The process of claim 20, comprising reacting the compound of formula (V) with hydrogen gas and the compound that provides a nitrogen-protecting group in the presence of the catalyst and a solvent.

22. The process of claim 20 or 21, wherein the compound that provides a nitrogen-protecting group is di-tert-butyldicarbonate.

23. The process of any one of claims 20-22, wherein the catalyst is Pd/C.

24. The process of claim 23, wherein the Pd/C is 5% Pd/C.

25. The process of any one of claims 21-24, wherein the solvent is methanol.

26. The process of any one of claims 20-25, wherein R³ is a resonance-accepting nitrogen-protecting group selected from tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps; and trifluoroacetyl.

27. The process of any one of claims 20-26, wherein R³ is tert-butyloxycarbonyl (Boc).

28. The process of any one of claims 12-27, wherein R² is C₁₋₆ alkyl.

29. The process of any one of claims 12-28, wherein R² is methyl.

30. The process of any one of claims 20-29, comprising:
reacting compound (1) with a compound that provides a nitrogen-protecting group, thereby producing a compound of formula (II): 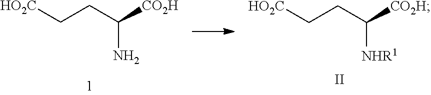
dehydrating the compound of formula (II), thereby producing a compound of formula (III): 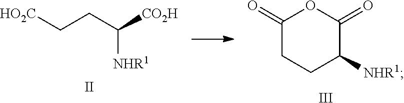
reacting the compound of formula (III) with compound (4), thereby producing a compound of formula (IV): 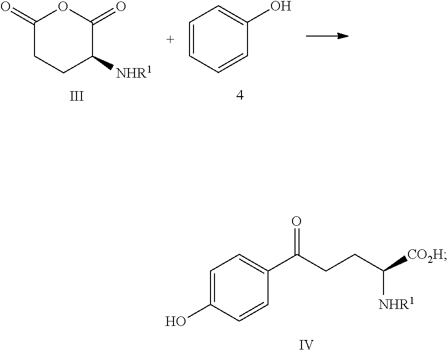
deprotecting the compound of formula (IV), thereby producing a compound of formula (V): 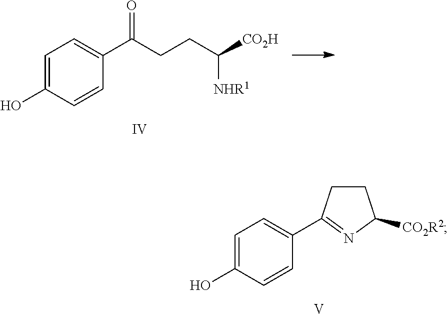
reacting the compound of formula (V) with hydrogen gas and the compound that provides a nitrogen-protecting group in the presence of the catalyst, thereby producing a compound of formula (VI): 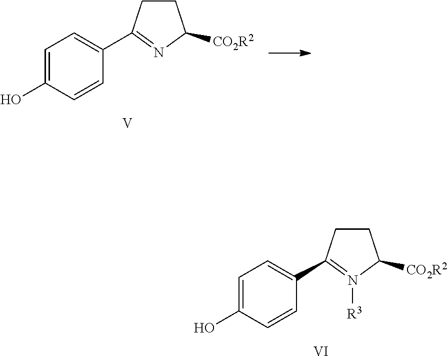
wherein:
R¹ is a resonance-accepting nitrogen-protecting group; and
R² is C₁₋₆alkyl, C₂₋₆ alkenyl, or C₂₋₆ alkynyl.

31. A compound of formula (IV) 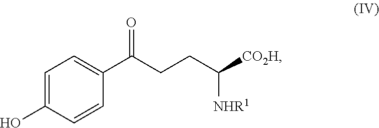
or a salt thereof, wherein R¹ is a resonance-accepting nitrogen-protecting group.

32. The compound of claim 31, wherein R¹ is a resonance-accepting nitrogen-protecting group selected from: tert-butyloxycarbonyl (Boc); 9-fluorenylmethyloxycarbonyl (Fmoc); acetyl (Ac); benzoyl (Bz); carbamates; tosyl (Ts); a sulfonamide selected from Nosyl and Nps; and trifluoroacetyl.

33. The compound of claim 31 or 32, wherein the compound of formula (IV) is compound (5), having the structure 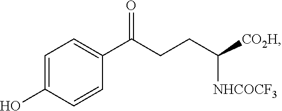
or a salt thereof.
